(19) United States
(12) Patent Application Publication
(10) Pub. No.: US 2006/0018849 A1
(43) Pub. Date: Jan. 26, 2006

(54) Cosmetic or dermatological preparations of the oil-in-water type

(75) Inventor: Anja Muller, Rumpel (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 11/191,230

(22) Filed: Jul. 27, 2005

Related U.S. Application Data
(63) Continuation of application No. 10/190,075, filed on Jul. 3, 2002, now abandoned.
Application No. 10/190,075 is a continuation of application No. PCT/EP00/12780, filed on Dec. 15, 2000.

(30) Foreign Application Priority Data
Jan. 5, 2000 (DE) 100 00 210.2

(51) Int. Cl. A61K 8/37

(52) U.S. Cl. 424/59

(57) ABSTRACT

The present invention includes cosmetic or dermatological preparations of the oil-in-water type comprising an oil phase having incorporated therein one or more citric esters of the general structural formula 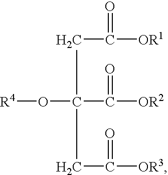
in which R¹, R² and R³, independently of one another, are chosen from the group consisting of ethyl, propyl and butyl, and R⁴ is a hydrogen atom or a CH₃(CO) group; and a water phase, where the difference in density between the oil phase and the water phase (determinable using a computerized digital density meter of the type DMA 45 from chempro/PA at 25° C.) is not greater than 0.01 g·cm⁻³. The present invention also includes a method for the stabilization of oil-in-water formulations.

### CROSS-REFERENCE TO RELATED APPLICATIONS

This is a continuation application of U.S. application Ser. No. 10/190,075, filed Jul. 3, 2002, which is a continuation application of PCT/EP00/12780, filed Dec. 15, 2000, and also claims the benefit of German Priority Application No. 100 00 210.2, filed Jan. 5, 2000, all of which are incorporated herein by reference in their entirety

### FIELD OF THE INVENTION

The present invention relates to cosmetic and dermatological preparations of the oil-in-water type, in particular flowable O/W emulsions, very particularly sprayable O/W emulsions, which have a viscosity of less than 2000 mPa·s, and to a method of stabilizing O/W formulations by matching the density of the phases.

### BACKGROUND OF THE INVENTION

Cosmetic preparations are essentially used for skincare. The human skin is the largest human organ and performs numerous vital functions. Having an average area of about 2 m² in adults, it has a prominent role as a protective and sensory organ. Amongst its many functions (for example for heat regulation), the barrier function, which prevents the skin (and therefore ultimately the entire organism) from drying out, is by far the most important. At the same time, the skin acts as a protective device against the invasion and the absorption of external substances (e.g. dirt, chemicals, microorganisms). In addition, it has an important role as a regulatory and target organ in human metabolism.

The main aim of cosmetic skincare is to strengthen or restore the skin's natural function as a barrier against environmental influences and against the loss of endogenous substances (as well as water also natural fats, electrolytes etc.).

Another aim of skincare is to compensate for the loss by the skin of lipids and water caused by daily washing. This is particularly important if the natural regeneration ability is inadequate. Furthermore, skincare products should protect against environmental influences, in particular against sun and wind, and delay skin aging.

The harmful effect of the ultraviolet part of solar radiation on the skin is generally known. Depending on their respective wavelength, the rays have different effects on the skin organ: UV-C radiation having a wavelength of less than 290 nm is absorbed by the ozone layer in the earth's atmosphere and is of no physiological importance. By contrast, rays in the range between 290 nm and 320 nm, the UV-B region, cause erythema, simple sunburn or even burns of varying severity. UV-A radiation (320 to 400 nm) is much more harmful than UV-B radiation with regard to the triggering of photodynamic, specifically phototoxic, reactions and chronic changes in the skin. For example, UV-A radiation on its own under very normal everyday conditions is enough to damage collagen and elastin fibers within a short period. The harmful effect of UV-B radiation can also be further intensified by UV-A radiation.

In addition, even very low radiation dosages can trigger photochemical reactions. These include, in particular, the formation of free radicals, which in turn can trigger uncontrolled secondary reactions as a result of their high reactivity. In order to prevent such reactions, as well as UV filter substances, it is also possible to additionally add antioxidants and/or free-radical scavengers to cosmetic or dermatological formulations.

Medicinal topical compositions usually comprise one or more medicaments in an effective concentration. For the sake of simplicity, in order to clearly distinguish between cosmetic and medicinal use and corresponding products, reference is made to the legal provisions of the Federal Republic of Germany (e.g. Cosmetics Directive, Foods and Drugs Act).

By far the most important type of product in the field of skincare compositions is emulsions. Emulsions are disperse two- or multi-phase systems, cosmetic emulsions consisting of at least one fatty phase (fats and mineral oils, fatty acid esters, fatty alcohols etc.) and at least one water phase (water, glycerol, glycols etc.), which are distributed in the form of very fine droplets in one another using emulsifiers. If the oil phase is finely distributed in the water phase, then this is an oil-in-water emulsion (O/W emulsion, e.g. milk). The basic character of an O/W emulsion is determined by the water, i.e. is less greasy on the skin, has more of a matting effect and absorbs more rapidly into the skin than a W/O emulsion.

Although, when viewed from a thermodynamic standpoint, emulsions are unstable systems, it is possible to prepare cosmetic emulsions which are stable for years. An emulsion is described as stable if, over a predefined period of time, no measurable temporal or spatial changes in the droplet size distribution can be established.

The stability or instability of emulsions depends on a variety of factors. Firstly, the water phase of a W/O emulsion tends, for example, toward sedimentation since the water and oil phases have different densities. The oil phase of an O/W emulsion, accordingly, has a tendency toward creaming.

In addition, because of the forces of attraction between the finely distributed droplets of the disperse phase, drop aggregation can result, where the individual droplets of an aggregate remain initially separate from one another by a thin film of the continuous phase. In this case, the original droplet size distribution only appears to change and can in this case be restored by stirring or shaking.

However, the droplets which are in contact can, moreover, also coalesce, which leads to a real change in the droplet size distribution, which can only be changed back by inputting energy. This phenomenon is referred to as coalescence. The more viscous the outer phase of the emulsion, the more slowly the process of coalescence proceeds.

The processes described can occur individually or together. One process often initiates or intensifies the other. Thus, for example, the formation of aggregates in O/W emulsions accelerates creaming of the oil phase. If the disperse state of an emulsion is partially or completely lost, then the two phases separate, and this is referred to as emulsion breaking.

Accordingly, the stabilization of emulsions over a relatively long period of time requires auxiliaries which prevent separation of the two phases, or at least delay it until the emulsion has fulfilled its intended purpose.

These auxiliaries should firstly stabilize the interface by preventing the droplets of the disperse phase from coalescing. In the ideal case, these substances moreover effect repulsion of the droplets, which prevents them from contacting, thus avoiding agglomeration (aggregate formation).

Secondly, auxiliaries are used to counteract creaming or sedimentation of the phases.

Emulsifiers are interface-active substances which are able to prevent the interfacial tension between oil and water phase by positioning themselves preferably at the interface between these two phases. This is made possible as a result of their amphiphilic molecular structure: emulsifiers have at least one polar (hydrophilic) group and at least one nonpolar (lipophilic) group. As a result, they are soluble both in the hydrophilic phase and in the lipophilic phase. The part which is more soluble in the corresponding phase protrudes into this phase and as a result lowers the interfacial tension between the two phases.

The attempt to classify emulsifiers is difficult since they belong to categories which are very different in chemical terms. The more quickly an emulsifier lowers the interfacial tension and the lower the equilibrium value of the interfacial tension, the more effective the emulsifier.

Moreover, emulsifiers also stabilize emulsions as a result of the formation of interfacial films and thus “physical” barriers, as a result of which aggregate formation and coalescence of the emulsified particles is prevented. As a result of the positioning of the emulsifier at the interface, the droplets either become charged, so that they mutually repel, or a stable, often high-viscosity or even solid protective layer is formed around the droplets.

However, for the practical preparation of cosmetic or dermatological emulsions, the use of one or more emulsifiers on their own is generally insufficient. Important factors for the stability of cosmetic or dermatological preparations are also:
very fine distribution of the two phases in one another
the smaller the dispersed particles, the more stable the emulsion.
high viscosity of the outer phase
a stable interfacial film
a balanced phase volume ratio

The emulsifier system must therefore in most cases comprise, in addition to the actual emulsifier, a further component which is referred to as coemulsifier, stabilizer or, depending on the activity mechanism, also as bodying agent, thickener or protective colloid etc.

These substances, which for the sake of simplicity are referred to below as stabilizers, increase the stability of an emulsion. Stabilizers do not have to be surface-active, but can be amphiphilically constructed compounds.

One way of stabilizing emulsions is, in accordance with what was stated above, to increase the viscosity of the external phase. This viscosity increase generally brings about a considerable reduction in the mobility of the dispersed droplets, as a result of which the rate of sedimentation or creaming is reduced. As a result of this, the droplets also meet less frequently, which results in a lower tendency toward coalescence.

The viscosity of the external phase can, for example, be increased by adding thickeners which form, for example, gels and/or lamellar liquid crystals. In principle, emulsifiers are also able to increase the viscosity of a liquid as a result of the formation of emulsifier gel networks. However, this requires a relatively large amount of emulsifier since gel networks are only formed when the total interface between the phases is coated with emulsifier molecules.

The breaking of an emulsion can also be prevented by the choice of a suitable phase volume ratio. To illustrate this fact, imagine an emulsion as a system of metal spheres of equal diameter (internal phase) and a liquid (external phase). Sedimentation or creaming can, in this simple model, no longer occur if the entire liquid is filled with metal spheres. Assuming as dense as possible a sphere packing as distribution, this is the case precisely at a ratio of 1:2, i.e. when ⅔ of the emulsion consists of an internal phase. It is obvious that the viscosity of an emulsion increases as the proportion of internal phase grows since the mobility of the dispersed droplets becomes restricted as a result.

The person skilled in the art is of course aware of the large number of options for formulating stable O/W preparations for cosmetic or dermatological use, for example in the form of creams and ointments, which are spreadable in the range from room to skin temperature, or as lotions and milks, which by contrast are flowable in this temperature range. In this connection, as well as the choice of the “right” emulsifier or emulsifier system, the further composition of the preparation, in particular, is important.

O/W emulsions are generally stabilized by thickeners which increase the viscosity of the aqueous phase. Examples of suitable thickeners for this purpose are polyacrylates (carbomers) and other organic thickeners. A disadvantage of this method of improving the stability is the sensitivity of these formulations toward electrolytes. In addition, by nature higher-viscosity formulations (such as creams or ointments) are of course to be prepared in this manner. The stabilization of O/W emulsions above the phase volume ratio also generally leads to viscous formulations, in accordance with what was stated above.

Emulsions of “liquid” (=flowable) consistency are used in cosmetics, for example as care, cleansing, face or hand lotions. They generally have a viscosity of from about 2 000 mPa·s to about 10 000 mPa·s. The stability of flowable emulsions requires particular attention since the considerably greater mobility of the particles encourages more rapid coalescence.

These prior art liquid emulsions—since they too generally comprise thickeners—are not stable toward relatively high concentrations of electrolyte either, which may manifest itself in phase separation. It is, however, frequently desirable to use certain electrolytes, such as, for example, water-soluble UV filters, in order to be able to utilize the other physical, chemical or physiological properties thereof. Although in many cases appropriate choice of the emulsifier system can provide remedies to a certain extent, other disadvantages then arise just as often.

The discussed disadvantages can, for example, lie in the fact that relatively large amounts of one or more emulsifiers are required (e.g. 3% by weight or above). Since, however, even emulsifiers—as ultimately any chemical substance—can in individual cases trigger allergic reactions or reactions based on oversensitivity of the user (although the use of customary cosmetic emulsifiers is of course generally entirely acceptable), it is desirable to keep the emulsifier content of a cosmetic or dermatological formulation as low as possible.

Emulsions with a very low viscosity (low-viscosity or sprayable emulsions) have hitherto, in accordance with what was stated above, only been able to be formulated with considerable effort, if at all. Accordingly, the supply of such formulations is extremely low. Nevertheless, such formulations could offer the consumer cosmetic results which were hitherto unknown.

Generally, low-viscosity preparations of the prior art frequently have the disadvantage that they are limited to a narrow area of application or a restricted choice of raw materials.

### SUMMARY OF THE INVENTION

An object of the present invention was to enrich the prior art with O/W preparations for cosmetic or dermatological use.

In addition, the object of the invention was to prepare preparations of the oil-in-water type which have a low or very low viscosity and do not have the disadvantages of the prior art. A further object of the invention was to discover ways of producing cosmetic or dermatological, as low-viscosity as possible, O/W emulsions which are stable toward increased electrolyte concentrations.

It was a further object of the present invention to find a method of stabilizing O/W formulations.

Surprisingly, these objects are achieved by cosmetic or dermatological preparations of the oil-in-water type, which comprise an oil phase into which one or more citric esters of the general structural formula 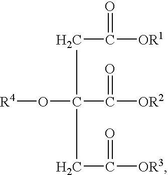
in which R¹, R² and R³, independently of one another, are chosen from the group consisting of ethyl, propyl and butyl, and R⁴ is a hydrogen atom or a CH₃(CO) group, are incorporated, and a water phase, where the difference in density between the oil phase and the water phase (determinable using a computerized digital density meter of the type DMA 45 from chempro/PA at 25° C.) is not greater than 0.01 g·cm⁻³ and, if desired, comprising cosmetic or dermatological auxiliaries, additives and/or active ingredients.

The invention further provides a method of stabilizing O/W formulations, which comprises matching the density of the oil phase to the density of the water phase by adding one or more citric esters of the general structural formula 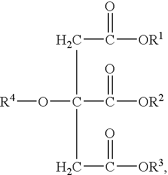
in which R¹, R² and R³, independently of one another, are chosen from the group consisting of ethyl, propyl and butyl, and R⁴ is a hydrogen atom or a CH₃(CO) group, in such a way that the difference in density between the two phases is not greater than 0.01 g·cm⁻³.

O/W formulations obtainable by this method are also provided by the present invention.

Advantageous citric esters for the purposes of the present invention are those for which R¹, R² and R³ are identical radicals. Triethyl citrate, acetyltriethyl citrate, tributyl citrate and acetyltributyl citrate are particularly advantageous. According to the invention, preference is also given to citric esters which are only sparingly soluble or even insoluble in water, i.e. particularly those for which at least one of the radicals R¹, R² and R³ is a butyl radical and/or for which R⁴ is an acetyl radical.

The preparations according to the invention are entirely satisfactory preparations in every respect and are not limited to a restricted choice of raw materials. Accordingly, they are particularly suitable for use as bases for preparation forms having diverse application purposes. The preparations according to the invention have excellent stability against decomposition in oil and water phases and exhibit very good sensory properties, such as, for example, spreadability on the skin or ability to be absorbed into the skin.

It was particularly surprising that the preparations according to the invention are extraordinarily stable even without addition of further stabilizers—such as, for example, bodying agents, thickeners or protective colloids etc. This could in no way have been foreseen, particularly for flowable or even sprayable formulations.

The preparations according to the invention represent an enrichment of the prior art in every respect with regard to O/W emulsions, in particular with regard to flowable or sprayable O/W emulsions.

In addition, very stable O/W formulations, for example sprayable formulations with a high sun protection factor, are obtainable by the method according to the invention in a surprisingly simple manner.

It is particularly advantageous according to the invention if the preparations comprise considerably less than 1% by weight (based on the total weight of the preparations) of one or more emulsifiers.

It is also advantageous according to the invention if the preparations have a viscosity of less than 10 000 mPa·s, more preferably less than 2 000 mPa·s, in particular less than 1 500 mPa·s (determinable using a Haake viscometer VT-02 at 25° C.).

It is also advantageous if the average diameter of the oil droplets of the formulations according to the invention is less than 50 μm. For the purposes of the present invention, preference is also given to formulations whose overall density is greater than 0.9 g·cm⁻³, in particular greater than 0.95 g·cm⁻³.

It may also be advantageous if the O/W formulations according to the invention, although it is not necessary, also comprise stabilizers, which are advantageously chosen from the group of thickeners. It is advantageous to choose the content of the thickener(s) from the range 0.05% by weight to 0.15% by weight, based on the total weight of the preparations.

The oil phase of the O/W formulations according to the invention advantageously has a high content of citrate esters according to the invention and may even consist entirely of such esters. Moreover, it may, however, also be advantageous to use further oils, for example in order to achieve a better feel of the formulations on the skin or to increase the performance of the products. The cosmetic and dermatological preparations according to the invention can, accordingly, also comprise oil components, as are customarily used in such preparations.

### DETAILED DESCRIPTION OF THE PREFERRED EMBODIMENTS

If the O/W formulations according to the present invention are to comprise emulsifiers, then it is advantageous to use those emulsifiers which are suitable for the preparation of O/W emulsions, it being possible for these to be present either individually or else in any combinations with one another.

The emulsifier(s) is/are advantageously chosen from the group consisting of the following compounds: polyglyceryl-2 dipolyhydroxystearate, PEG-30 dipolyhydroxystearate, cetyl-dimethicone copolyol, glycol distearate, glycol dilaurate, diethylene glycol dilaurate, sorbitan trioleate, glycol oleate, glyceryl dilaurate, sorbitan tristearate, propylene glycol stearate, propylene glycol laurate, propylene glycol distearate, sucrose distearate, PEG-3 castor oil, pentaerythrityl monostearate, pentaerythrityl sesquioleate, glyceryl oleate, glyceryl stearate, glyceryl diisostearate, pentaerythrityl monooleate, sorbitan sesquioleate, isostearyl diglyceryl succinate, glyceryl caprate, palm glycerides, cholesterol, lanolin, glyceryl oleate (containing 40% monoester), polyglyceryl-2 sesquiisostearate, polyglyceryl-2 sesquioleate, PEG-20 sorbitan beeswax, sorbitan oleate, sorbitan isostearate, trioleyl phosphate, glyceryl stearate and ceteareth-20 (Teginacid from Th. Goldschmidt), sorbitan stearate, PEG-7 hydrogenated castor oil, steareth-2, oleth-2, cetyl alcohol and ceteareth-30 (emulsifier E 2209 from Th. Goldschmidt), PEG-5 soya sterol, PEG-6 sorbitan beeswax, ceteth-2, glyceryl stearate SE, methylglucose sesquistearate, PEG-10 hydrogenated castor oil, oleth-3, sorbitan palmitate, PEG-22/dodecyl glycol copolymer, polyglyceryl-2 PEG-4 stearate, laneth-5, ceteth-3, laureth-3, stearyl alcohol and steareth-7 and steareth-10 (emulsifier E-2155 from Th. Goldschmidt), oleth-5, sorbitan laurate, laureth-4, PEG-4 laurate, polysorbate 61, polysorbate 81, beheneth-10, polysorbate 65, polysorbate 80, laneth-10, triceteareth-4 phosphate, triceteareth-4 phosphate and sodium C₁₄₋₁₇-alkyl sec sulfonate (Hostacerin CG from Hoechst), PEG-8 stearate, glyceryl stearate and PEG-100 stearate (Arlacel 165 from ICI), polysorbate 85, trilaureth-4 phosphate, PEG-25 glyceryl trioleate, oleth-10, steareth-10, ceteth-10, PEG-35 castor oil, sucrose stearate, PEG-8 oleate, trioleth-8 phosphate, PEG-40 sorbitan lanolate, PEG-15 glyceryl ricinoleate, choleth-24 and ceteth-24 (Solulan C-24 from Amerchol), C₁₂₋₁₅-pareth-12, PEG-20 glyceryl isostearate, PEG-40 hydrogenated castor oil, PEG-16 soya sterol, PEG-20 glyceryl oleate, PEG-20 stearate, polysorbate 80, PEG-20 methylglucose sesquistearate, PEG-30 glyceryl isostearate, PEG-20 glyceryl laurate, ceteth-20, ceteareth-25, PEG-30 stearate, PEG-30 glyceryl stearate, polysorbate 20, laureth-23, PEG-40 stearate, PEG-30 glyceryl laurate, PEG-50 stearate, PEG-100 stearate, PEG-150 laurate, polyglyceryl-3 methylglucose distearate, ceteareth-12, ceteareth-20 and steareth-21, ceteareth-6, PEG-40 castor oil, sodium cetearyl sulfate, lecithin, laureth-4 phosphate, propylene glycol stearate SE, PEG-25 hydrogenated castor oil, PEG-54 hydrogenated castor oil, glyceryl stearate SE, PEG-6 caprylic/capric glycerides, glyceryl oleate and propylene glycol, PEG-9 stearate, glyceryl lanolate, ceteth-2, polysorbate 60, glyceryl myristate, glyceryl isostearate and polyglyceryl-3 oleate, glyceryl laurate, PEG-40 sorbitan peroleate, laureth-4, glycerol monostearate, ceteareth-3, lanolin acid, isostearyl glyceryl ether, cetearyl alcohol and sodium cetearyl sulfate, steareth-2, PEG-22 dodecyl glycol copolymer, polyglyceryl-2 PEG-4 stearate, pentaerythrityl isostearate, polyglyceryl-3 diisostearate, sorbitan oleate and hydrogenated castor oil and Cera alba and stearic acid, sodium dihydroxycetyl phosphate and isopropyl hydroxycetyl ether, methylglucose sesquistearate, steareth-2 and PEG-8 distearate, steareth-20, isosteareth-20, methylglucose dioleate, sorbitan oleate and PEG-2 hydrogenated castor oil and ozokerite and hydrogenated castor oil, PEG-2 hydrogenated castor oil, PEG-45/dodecyl glycol copolymer, methoxy PEG-22/dodecyl glycol copolymer, hydrogenated cocoglycerides, polyglyceryl-4 isostearate, PEG-40 sorbitan peroleate, PEG-40 sorbitan perisostearate, PEG-20 glyceryl stearate, PEG-8 beeswax, laurylmethicone copolyol, polyglyceryl-2 laurate, stearamidopropyl PG dimonium chloride phosphate, PEG-7 hydrogenated castor oil, triethyl citrate, PEG-20 methylglucose sesquistearate, glyceryl stearate citrate, cetyl phosphate, polyglycerol methylglucose distearate, poloxamer 101, potassium cetyl phosphate, isosteareth-10, oleth-20, isoceteth-20, glyceryl isostearate, polyglyceryl-3 diisostearates, cetearyl alcohol and PEG-20 stearate.

The emulsifier(s) is/are particularly preferably chosen from the group of fatty acids which have been completely or partially neutralized with customary alkalis (such as, for example, sodium and potassium hydroxide, sodium and potassium carbonate, and mono- and triethanolamine). Particularly advantageous examples are stearic acid and stearates, isostearic acid and isostearates, palmitic acid and palmitates, and myristic acids and myristates.

According to the invention, the emulsifiers are also preferably chosen from the group of saturated and/or unsaturated, branched and/or unbranched fatty alcohols having 10 to 40 carbon atoms, particular preference being given to butyloctanol, butyldecanol, hexyloctanol, hexyldecanol, octyldodecanol, behenyl alcohol (C₂₂H₄₅OH), cetearyl alcohol [a mixture of cetyl alcohol (C₁₆H₃₃OH) and stearyl alcohol (C₁₈H₃₇OH)], cetyl arachidol [2-hexadecyl-1-eicosanol (C₃₆H₇₃OH)], lanolin alcohols (wool wax alcohols which are the non-hydrolyzable alcohol fraction of wool wax which is obtained after the hydrolysis of wool wax) and/or 2-tetradecyloctadecanol (C₃₂H₆₅OH). Advantageous variants of the two last-mentioned fatty alcohols are available under the trade names Isofol 36 and Isofol 32 from Condea.

The list of emulsifiers mentioned which can be used for the purposes of the present invention is of course not intended to be limiting.

The preparations according to the invention can advantageously also comprise one or more hydrocolloids.

Hydrocolloids are macromolecules which have a largely linear structure and have intermolecular forces of interaction, which permit secondary and primary valence bonds between the individual molecules and thus the formation of a reticular structure. Some are water-soluble natural or synthetic polymers which, in aqueous systems, form gels or viscous solutions. They increase the viscosity of the water by either binding water molecules (hydration) or else by absorbing and encapsulating the water into their interwoven macromolecules, at the same time restricting the mobility of the water.

The group of hydrocolloids can be divided as follows into:
organic, natural compounds, such as, for example, agar agar, carrageen, tragacanth, gum arabic, alginates, pectins, polyoses, guar gum, carob bean flour, starch, dextrins, gelatins, casein,
organic, modified natural substances, such as, for example, carboxymethylcellulose and other cellulose ethers, hydroxyethyl- and -propylcellulose and the like,
organic, completely synthetic compounds, such as, for example, polyacrylic and polymethacrylic compounds, vinyl polymers, polycarboxylic acids, polyethers, polyimines, polyamides,
inorganic compounds, such as, for example, polysilicic acids, clay minerals, such as montmorillonites, zeolites, silicas.

Examples of hydrocolloids which are preferred according to the invention are methylcelluloses, which is the name for the methyl ethers of cellulose. They are characterized by the following structural formula 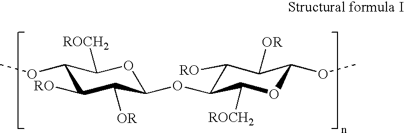
in which R can be a hydrogen or a methyl group.

Particularly advantageous for the purposes of the present invention are the cellulose mixed ethers, which are generally likewise referred to as methylcelluloses, which contain, in addition to a dominating content of methyl groups, also 2-hydroxyethyl, 2-hydroxypropyl or 2-hydroxybutyl groups. Particular preference is given to (hydroxypropyl)methylcelluloses, for example those available under the trade name Methocel E4M from Dow Chemical Comp.

Also advantageous according to the invention is sodium carboxymethylcellulose, the sodium salt of the glycolic ether of cellulose, for which R in structural formula I can be a hydrogen and/or CH₂—COONa. Particular preference is given to the sodium carboxymethylcellulose available under the trade name Natrosol Plus 330 CS from Aqualon and also referred to as cellulose gum.

Also preferred for the purposes of the present invention is xanthan (CAS No. 11138-66-2), also called xanthan gum, which is an anionic heteropolysaccharide which is generally formed by fermentation from maize sugar and is isolated as the potassium salt. It is produced by Xanthomonas campestris and a number of other species under aerobic conditions with a molecular weight of 2×10⁶ to 24×10⁶. Xanthan is formed from a chain having β-1,4-bonded glucose (cellulose) with side chains. The structure of the subgroups (“repeat units”) consists of glucose, mannose, glucuronic acid, acetate and pyruvate.

Other hydrocolloids which are advantageous according to the invention are polymers of acrylic acid, in particular those chosen from the group of carbomers or Carbopols (Carbopol® is actually a registered trade mark of the B. F. Goodrich Company). Carbopols are compounds of the general structural formula 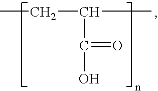
whose molecular weight can be between about 400 000 and more than 4 000 000. The group of Carbopols also includes acrylate-alkyl acrylate copolymers, for example those characterized by the following structure: 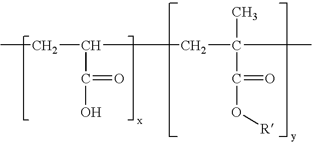
where R′ is a long-chain alkyl radical, and x and y are numbers which symbolize the respective stoichiometric content of each comonomer. These Carbopols, too, are also advantageous for the purposes of the present invention.

Examples of advantageous Carbopols are the grades 907, 910, 934, 940, 941, 951, 954, 980, 981, 1342, 1382, 2984 and 5984, it being possible for these compounds to be present individually or in any combinations with one another. Particular preference is given to Carbopol 981, 1382 and 5984 (either individually or in combination with other hydrocolloids).

Also advantageous for the purposes of the present invention are the copolymers, comparable with the acrylate-alkyl acrylate copolymers, of C₁₀₋₃₀-alkyl acrylates and one or more monomers of acrylic acid, of methacrylic acid or esters thereof. The INCI name for such compounds is “Acrylates/C 10-30 Alkyl Acrylate Crosspolymer”. Particularly advantageous are those available under the trade names Pemulen TR1 and Pemulen TR2 from B. F. Goodrich Company.

The total amount of one or more hydrocolloids in the finished cosmetic or dermatological preparations is advantageously chosen to be less than 1.0% by weight, preferably between 0.01 and 0.5% by weight, based on the total weight of the preparations.

The aqueous phase of the preparations according to the invention optionally advantageously comprises alcohols, diols or polyols of low carbon number and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, preferably ethanol, isopropanol, 1,2-propanediol and glycerol.

Particularly advantageous preparations are also obtained when antioxidants are used as additives or active ingredients. According to the invention, the preparations advantageously comprise one or more antioxidants. Favorable, but nevertheless optional antioxidants to be used are all antioxidants suitable or customary for cosmetic and/or dermatological applications.

The antioxidants are advantageously chosen from the group consisting of amino acids (e.g. glycine, histidine, tyrosine, tryptophan) and derivatives thereof, imidazoles, (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximines) in very low tolerated doses (e.g. pmol to μmol/kg), and also (metal) chelating agents (e.g. α-hydroxyfatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin resin, rutinic acid and derivatives thereof, ferulic acid and derivatives thereof, butyl hydroxytoluene, butyl hydroxyanisole, nordihydroguaiacic acid, nordihydroguaiaretic acid, trihydroxy-butyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide), and the derivatives (salts, esters, ethers, sugars, nucleotides, nucleosides, peptides and lipids) of said active substances which are suitable according to the invention.

For the purposes of the present invention, the use of oil-soluble antioxidants is particularly advantageous.

A surprising property of the present invention is that preparations according to the invention are very good vehicles for cosmetic or dermatological active ingredients into the skin, preferred active ingredients being antioxidants which can protect the skin from oxidative stress. Preferred antioxidants in this connection are vitamin E and derivatives thereof, and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant(s), it is advantageous to choose their respective concentrations from the range 0.001 to 10% by weight, based on the total weight of the formulation.

If vitamin A or vitamin A derivatives, or carotenes or derivatives thereof are the antioxidant(s), it is advantageous to choose their respective concentrations from the range 0.001 to 10% by weight, based on the total weight of the formulation.

The person skilled in the art is of course aware that cosmetic preparations are in most cases inconceivable without customary auxiliaries and additives. The cosmetic and dermatological preparations according to the invention can, accordingly, also comprise cosmetic auxiliaries as are customarily used in such preparations, for example bodying agents, fillers, preservatives, perfumes, antifoams, dyes, other surface-active substances, emolients, moisturizers and/or humectants, anti-inflammatory substances, additional active ingredients, such as vitamins or proteins, light protection agents, insect repellents, bactericides, virusides, water, salts, antimicrobial, proteolytic or keratolytic substances, medicaments or other customary constituents of a cosmetic or dermatological formulation, such as alcohols, polyols, polymers, foam stabilizers, organic solvents and also electrolytes.

Corresponding requirements apply mutatis mutandis to the formulation of medicinal preparations.

The O/W emulsions according to the invention can be used as a basis for cosmetic and dermatological formulations. The latter can have the customary composition and be used, for example, for the treatment and care of the skin and/or the hair, as a lipcare product, as a deodorant product and as make-up or a make-up remover product in decorative cosmetics or as a light protection preparation. For use, the cosmetic and dermatological preparations according to the invention are applied to the skin and/or hair in a sufficient amount in a manner customary for cosmetics or dermatological compositions.

Accordingly, for the purposes of the present invention, cosmetic or topical dermatological compositions can, depending on their composition, be used, for example, as skin protection cream, cleansing milk, sunscreen lotion, nutrient cream, day or night cream etc. In some instances, it is possible and advantageous to use the compositions according to the invention a basis for pharmaceutical formulations.

The low-viscosity cosmetic or dermatological preparations according to the invention can, for example, be in the form of preparations which can be sprayed from aerosol containers, squeezable bottles or by means of a pump device, or in the form of a liquid composition which can be applied by means of roll-on devices, but also in the form of an emulsion which can be applied from normal bottles and containers.

For the purposes of the present invention, suitable propellants for cosmetic or dermatological preparations which can be sprayed from aerosol containers are the customary known readily volatile, liquefied propellants, for example hydrocarbons (propane, butane, isobutane), which can be used alone or in a mixture with one another. Compressed air is also used advantageously.

The person skilled in the art is of course aware that there are propellant gases which are nontoxic per se which would be suitable in principle for realizing the present invention in the form of aerosol preparations, but which must nevertheless be avoided because of their harmful impact on the environment or other accompanying circumstances, in particular fluorinated hydrocarbons and chlorofluorocarbons (CFCs).

Those cosmetic and dermatological preparations which are in the form of a sunscreen are also favorable. These preferably additionally comprise at least one UV-A filter substance and/or at least one UV-B filter substance.

It is, however, also advantageous for the purposes of the present invention to provide those cosmetic and dermatological preparations whose main purpose is not protection against sunlight, but which nevertheless have a content of UV protection substances. Thus, for example, UV-A and/or UV-B filter substances are usually incorporated into day creams.

UV protection substances, like antioxidants and, if desired, preservatives, also represent effective protection of the preparations themselves against decay.

Preparations according to the invention advantageously comprise substances which absorb UV radiation in the UV-A and/or UV-B region, where the total amount of the filter substances is, for example, 0.1% by weight to 30% by weight, preferably 0.5 to 20% by weight, in particular 1.0 to 15.0% by weight, based on the total weight of the preparations, in order to provide cosmetic preparations which protect the hair and/or the skin from the entire range of ultraviolet radiation. They can also be used as sunscreens for the hair or the skin.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.

Further advantageous UV-A filter substances are phenylene-1,4-bis(2-benzimidazyl)-3,3′-5,5′-tetrasulfonic acid: 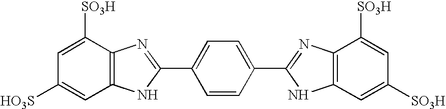
and its salts, particularly the corresponding sodium, potassium or triethanolammonium salts, in particular phenylene-1,4-bis(2-benzimidazyl)-3,3′-5,5′-tetrasulfonic bis-sodium salt: 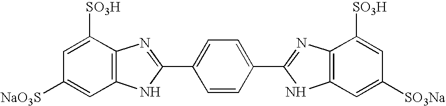
and 1,4-di(2-oxo-10-sulfo-3-bomylidenemethyl)benzene and salts thereof (in particular the corresponding 10-sulfato compounds, in particular the corresponding sodium, potassium or triethanolammonium salt), which is also referred to as benzene-1,4-di(2-oxo-3-bomylidenemethyl-10-sulfonic acid) and is characterized by the following structure: 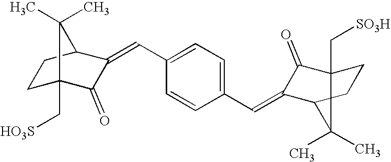
Advantageous UV filter substances for the purposes of the present invention are also “broad-band” filters, i.e. filter substances which absorb both UV-A and also UV-B radiation.

Advantageous broad-band filters or UV-B filter substances are, for example, bisresorcinyltriazine derivatives having the following structure: 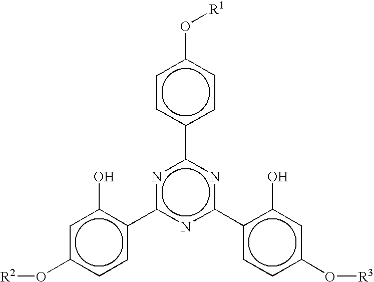
where R¹, R² and R³, independently of one another, are chosen from the group of branched and unbranched alkyl groups having 1 to 10 carbon atoms, or are a single hydrogen atom. Particular preference is given to 2,4-bis{[4-(2-ethylhexyloxy)-2-hydroxy]phenyl}-6-(4-methoxyphenyl)-1,3,5-triazine (INCI: Aniso Triazine), which is available under the trade name Tinosorb® S from CIBA-Chemikalien GmbH and to tris(2-ethylhexyl) 4,4′,4″-(1,3,5-triazine-2,4,6-triyltriimino)trisbenzoate, synonym: 2,4,6-tris[anilino(p-carbo-2′-ethyl-1′-hexyloxy)]-1,3,5-triazine (INCI: Octyl Triazone), which is marketed by BASF Aktiengesellschaft under the trade name UVINUL® T 150.

Other UV filter substances, which have the structural formula 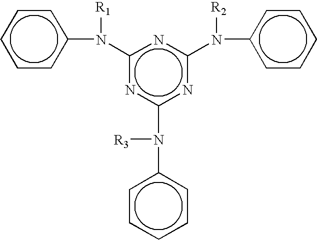
are also advantageous UV filter substances for the purposes of the present invention, for example the s-triazine derivatives described in European laid-open specification EP 570 838 A1, whose chemical structure is expressed by the generic formula 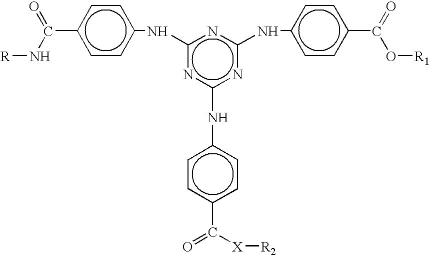
where
R is a branched or unbranched C₁-C₁₈-alkyl radical, a C₅-C₁₂-cycloalkyl radical, optionally substituted with one or more C₁-C₄-alkyl groups,
X is an oxygen atom or an NH group,
R₁ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 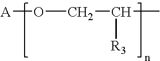
   in which
   A is a branched or unbranched C₁-C₁₈-alkyl radical, a C₅-C₁₂-cycloalkyl or aryl radical, optionally substituted by one or more C₁-C₄-alkyl groups,
   R₃ is a hydrogen atom or a methyl group,
   n is a number from 1 to 10,
R₂ is a branched or unbranched C₁-C₁₈-alkyl radical, a C₅-C₁₂-cycloalkyl radical, optionally substituted by one or more C₁-C₄-alkyl groups, when X is the NH group, and
   a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 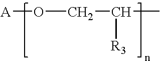
   in which
   A is a branched or unbranched C₁-C₁₈-alkyl radical, a C₅-C₁₂-cycloalkyl or aryl radical, optionally substituted by one or more C₁-C₄-alkyl groups,
   R₃ is a hydrogen atom or a methyl group,
   n is a number from 1 to 10,
   when X is an oxygen atom.

A particularly preferred UV filter substance for the purposes of the present invention is also an unsymmetrically substituted s-triazine, the chemical structure of which is expressed by the formula 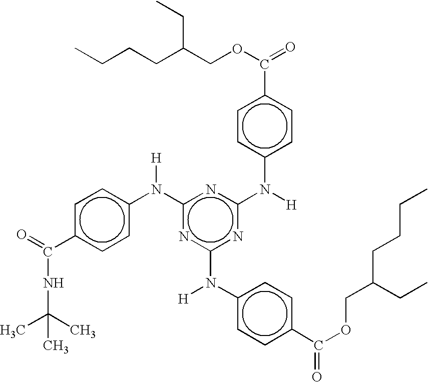
and which is also referred to below as dioctylbutylamidotriazone. European laid-open specification 775 698 also describes preferred bisresorcinyltriazine derivatives to be used, the chemical structure of which is expressed by the generic formula 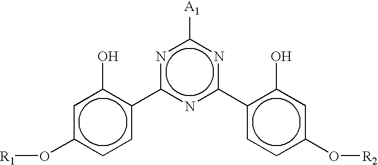
where R₁, R₂ and A₁ represent very different organic radicals.

Also advantageous for the purposes of the present invention are 2,4-bis{[4-(3-sulfonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl}-6-[4-(2-methoxyethylcarboxyl)phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1-methylpyrrol-2-yl)-1,3,5-triazine, 2,4-bis{[4-tris(trimethylsiloxysilylpropyloxy)-2-hydroxy]phenyl}-6-(4-methoxyphenyl)-1,3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

An advantageous broad-band filter for the purposes of the present invention is 2,2′-methylenebis(6-(2H-benzotriazol-2-yl)-4-(1,1,3,3-tetramethylbutyl)phenol), which is characterized by the chemical structural formula 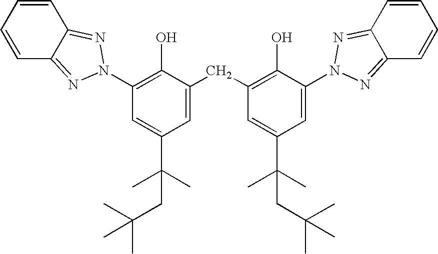
and is available under the trade name Tinosorb® M from CIBA-Chemikalien GmbH.

Another advantageous broad-band filter for the purposes of the present invention is 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) having the INCI name Drometrizole Trisiloxane, which is characterized by the chemical structural formula

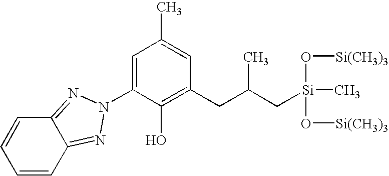

The UV-B filters can be oil-soluble or water-soluble. Examples of advantageous oil-soluble UV-B filter substances are:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
2,4,6-trianilino(p-carbo-2′-ethyl-1′-hexyloxy)-1,3,5-triazine;
esters of benzalmalonic acid, preferably di(2-ethylhexyl) 4-methoxybenzalmalonate,
esters of cinnamic acid, preferably 2-ethylhexyl 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone
and UV filters bonded to polymers.

Examples of advantageous water-soluble UV-B filter substances are:
salts of 2-phenylbenzimidazole-5-sulfonic acid, such as its sodium, potassium or its triethanolammonium salt, and also the sulfonic acid itself;
sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

A further light protection filter substance which can be used advantageously according to the invention is ethylhexyl 2-cyano-3,3-diphenyl acrylate (octocrylene), which is available from BASF under the name Uvinul® N 539 and is characterized by the following structure:

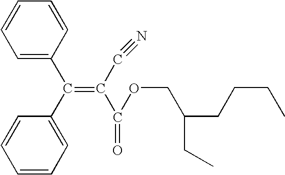

It can also be of considerable advantage to use polymer-bonded or polymeric UV filter substances in the preparations according to the present invention, in particular those described in WO-A-92/20690.

In some instances, it may also be advantageous according to the invention to incorporate further UV-A and/or UV-B filters into cosmetic or dermatological preparations, for example certain salicylic acid derivatives, such as 4-isopropylbenzyl salicylate, 2-ethylhexyl salicylate (=octyl salicylate), homomenthyl salicylate.

The list of said UV filters which can be used for the purposes of the present invention is of course not intended to be limiting.

The examples below serve to illustrate the present invention without limiting it. The numerical values in the examples are percentages by weight, based on the total weight of the respective preparations.

### EXAMPLES

| | 1 | 2 | 3 | 4 | 5 | 6 |
|---|---|---|---|---|---|---|
| Glyceryl stearate citrate | 0.5 | 0.5 | | 0.5 | 0.5 | |
| Glyceryl stearate | | | 0.5 | | | 0.5 |
| Tributyl citrate | 10 | | 5 | 5 | | 3 |
| Acetyltributyl citrate | | 10 | | 5 | 5 | 3 |
| Caprylic/capric | | 2 | | 2 | | |
| triglycerid | | | | | | |
| C₁₂₋₁₅-alkyl benzoate | | 2 | 5 | 2 | 3 | |
| Butylene glycol | | 2 | 5 | 2 | | |
| dicaprylate/dicaprate | | | | | | |
| Octyltriazone | | | 0.5 | | | |
| Methylbenzylidenecamphor | | | 2 | | | |
| Butylmethoxydibenzoyl- | | | 2 | | | |
| methane | | | | | | |
| Titanium dioxide | | | | 3 | | |
| Vitamin E acetate | | | 0.5 | 0.5 | | |
| Preservative | 0.7 | 0.7 | 0.7 | 0.7 | 0.7 | 0.7 |
| Xanthan gum | | | | 0.1 | 0.1 | |
| Carbomer | | 0.1 | | | | 0.05 |
| Glycerol | 10 | 1.6 | 10 | 9 | | 10 |
| NaCl | | | | | | 3.8 |
| Sodium | 3.5 | | 8 | | | |
| phenylbenzimidazole- | | | | | | |
| sulfonate | | | | | | |
| Water | 75.3 | 81.1 | 60.8 | 70.2 | 90.7 | 78.95 |

1. A cosmetic or dermatological oil-in-water preparation, comprising an oil phase having incorporated therein one or more citric esters of the general structural formula 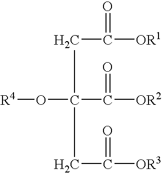
in which R¹, R² and R³, independently of one another, are selected from the group consisting of ethyl, propyl and butyl, and R⁴ is a hydrogen atom or a CH₃(CO) group; and a water phase, wherein the difference in density between the oil phase and the water phase as determined using a computerized digital density meter DMA 45 from Chempro/PA at 25° C. is no greater than 0.01 g·cm⁻³ and wherein the content of emulsifiers is less than 1% by weight, based on the total weight of the preparation.

2. The preparation as claimed in claim 1, wherein the one or more citric esters are selected from the group consisting of triethyl citrate, acetyltriethyl citrate, tributyl citrate and acetyltributyl citrate.

3. The preparation as claimed in claim 1, further comprising one or more compounds selected from the group consisting of cosmetic or pharmaceutical auxiliaries, additives and active ingredients.

4. The preparation as claimed in claim 3, wherein the cosmetic or pharmaceutical auxiliaries, additives and active ingredients include one or more antioxidants.

5. The preparation as claimed in claim 3, wherein the cosmetic or pharmaceutical auxiliaries, additives and active ingredients include one or more UV protection substances.

6. The preparation as claimed in claim 1, wherein the viscosity of the preparation is less than 1500 mPa·s.

7. The preparation as claimed in claim 1, wherein the preparation is sprayable.

8. The preparation as claimed in claim 1, wherein the diameter of the oil droplets is, on average, less than 50 μm.

9. The preparation as claimed in claim 1, wherein the density of the overall formulation is greater than 0.9 g·cm⁻³.

10. The preparation as claimed in claim 1, wherein the density of the overall formulation is greater than 0.95 g·cm⁻³.

11. The preparation as claimed in claim 1, wherein the content of thickeners is between 0.05% by weight and 0.15% by weight, based on the total weight of the preparation.

12. The preparation as claimed in claim 1, wherein the preparation is in the form of a treatment and care composition for the hair or the skin, a lipcare composition, a deodorant composition, a make-up composition or a make-up remover composition.

13. A cosmetic or dermatological light protection preparation comprising the preparation as claimed in claim 1 and one or more light protection agents.

14. The preparation as claimed in claim 1, wherein said one or more citric esters are present in an amount sufficient to stabilize said preparation without additional stabilizers.

15. A method of using an oil-in-water emulsion comprising an oil phase having incorporated therein one or more citric esters of the general structural formula 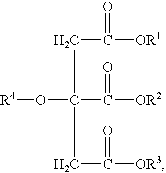
in which R¹, R² and R³, independently of one another, are selected from the group consisting of ethyl, propyl and butyl, and R⁴ is a hydrogen atom or a CH₃(CO) group; and a water phase, wherein the difference in density between the oil phase and the water phase as determined using a computerized digital density meter DMA 45 from Chempro/PA at 25° C. is no greater than 0.01 g·cm⁻¹, wherein the content of emulsifiers is less than 1% by weight, based on the total weight of the preparation, as a cosmetic or dermatological preparation comprising the step of applying a composition that includes the oil-in-water emulsion to one or more of the skin and the hair.

16. The method as claimed in claim 15, wherein said one or more citric esters are present in an amount sufficient to stabilize said preparation without additional stabilizers.

17. The preparation as claimed in claim 1, wherein the viscosity of the preparation is less than 2000 mPa·s.

18. The preparation as claimed in claim 1, further comprising one or more hydrocolloids.

19. The method as claimed in claim 15, wherein the viscosity of the preparation is less than 2000 mPa·s.

20. The method as claimed in claim 15, wherein the preparation further comprises one or more hydrocolloids.
